(19) United States
(12) Patent Application Publication
(10) Pub. No.: US 2013/0109017 A1
(43) Pub. Date: May 2, 2013

(54) MULTIPLE MYELOMA PROGNOSIS AND TREATMENT

(71) Applicant: University of Utah Research Foundation, Salt Lake City, UT (US)

(72) Inventors: Maurizio Zangari, Salt Lake City, UT (US); Fenghuang ZHAN, Salt Lake City, UT (US); Guido TRICOT, Salt Lake City, UT (US)

(73) Assignee: University of Utah Research Foundation, Salt Lake City, UT (US)

(21) Appl. No.: 13/717,264

(22) Filed: Dec. 17, 2012

Related U.S. Application Data
(62) Division of application No. 13/293,880, filed on Nov. 10, 2011, now abandoned.
(60) Provisional application No. 61/412,880, filed on Nov. 12, 2010.

(51) Int. Cl. C12Q 1/68; G01N 33/68

(52) U.S. Cl. CPC C12Q 1/6886; G01N 33/6872
USPC 435/6.11; 435/7.23; 435/6.14; 435/6.12

(57) ABSTRACT

Disclosed herein are diagnostic and prognostic methods for determining the overall survival, and therapeutic regimes, for multiple myeloma patients. The methods involve the detection of PTHR1 gene expression alone or in combination with other clinical factors. The tests are suitable for diagnosing and monitoring treatment of patients having or suspected of having multiple myeloma. The disclosure also relates to proteasome inhibitors and other activators of PTHR1, for the treatment of multiple myeloma.

### CROSS-REFERENCE TO RELATED APPLICATIONS

This application is a Divisional of U.S. patent application Ser. No. 13/293,880, filed Nov. 10, 2011, which claims priority to U.S. Provisional Patent Application No. 61/412,880, filed Nov. 12, 2010, both of which are incorporated herein by reference in their entirety.

### TECHNICAL FIELD

The present disclosure relates generally to the diagnosis, prognosis, treatment, and management of disease, including cancer. In particular, the present disclosure relates to methods for detecting and analyzing gene expression profiles associated with multiple myeloma, and administering suitable treatments therefor.

### BACKGROUND

The following discussion of the background is merely provided to aid the reader in understanding the present disclosure and is not admitted to describe or constitute prior art.

Multiple myeloma (MM) is a plasma cell malignancy with high osteolytic capacity and impaired bone formation. Typically, bone disease is comorbid with a diagnosis of MM and is associated with bone pain, fractures, spinal cord compression and hypercalcemia of malignancy. The pathogenesis of bone disease is complex. Numerous biological processes and pathways have been implicated in maintaining the balance between osteoblast bone formation and the bone-resorptive activity of osteoclasts. In MM bone disease, osteoclastic activity is increased through various myeloma-cell interactions in bone marrow, which results in a substantial increased in bone resorption. Moreover, osteoblast differentiation is impaired in MM patients, thereby curtailing bone formation. The combined effects of increased bone resorption with stymied bone formation is deleterious for bone vitality, and compounds the nature of MM. Such bone impairments, however, are only part of the pathological penumbras surrounding MM.

MM is currently an incurable disease with survival rates that range from a few months to more than 15 years. It is estimated that patients with myeloma have 10¹²-10¹³ myeloma cells and at complete remission may still have up to 10⁹ myeloma cells present. Since tumor burden does not appear to be the major prognostic marker in multiple myeloma, achieving a prolonged event free survival is not likely dependent upon an absolute reduction in myeloma burden, but rather on genetic characteristics of the patient. In fact, in many different types of malignancies, achievement of a complete remission and eradication of all macroscopic disease has not resulted in the improvement of overall survival rates.

The majority of MM tumor cells can be killed by conventional chemotherapies and cancer cells affected by those therapies have a limited proliferative potential. Nevertheless, patients harboring genetic aberrations, or that lack induction of oncogenic regulators, may be less responsive to chemotherapeutic agents than patients without such characteristics. These genetic factors may be present at diagnosis or, alternatively, may emanate from exposure to various chemotherapies. As such, identification of individuals that may be responsive or resistant to particular therapies would improve their oncological assessment at the time of a diagnosis.

### SUMMARY

In one aspect, the present disclosure generally describes methods for determining a diagnosis, prognosis, or treatment regime for multiple myeloma in a subject by detecting a level of PTHR1 expression in a test sample from the subject, wherein a difference in the level of PTHR1 expression in the subject compared to a reference level is an indication of the subject's responsiveness to therapy, including one or more proteasome inhibitors, PTH therapy, and PTH analog therapy, or any combination thereof. In one embodiment, the difference is an increase in the level of PTHR1 expression in the subject compared to the reference level, and the increase indicates that the multiple myeloma is susceptible to the therapy selected from one or more proteasome inhibitors, PTH, or PTH analogs, or any combination thereof.

In one embodiment, the one or more proteasome inhibitors is Bortezomib, Disulfuram, Salinosporamide A, Carfilzomib, CEP-18770, or MLN9708, or any combination thereof. In one embodiment, the difference is an increase in the level of PTHR1 expression in the subject compared to the reference level, and the increase is prognostic for an improved overall survival of the subject undergoing the therapy, compared to individuals afflicted with multiple myeloma that do not have the increase in the level of PTHR1.

In one embodiment, the difference is a decrease in the level of PTHR1 expression in the subject compared to the reference level and the decrease indicates that the multiple myeloma is resistant to therapy, including one or more proteasome inhibitors, PTH therapy, and PTH analog therapy, or any combination thereof. In one embodiment, the difference is a decrease in the level of PTHR1 expression in the subject compared to the reference level and the decrease is prognostic for a diminished overall survival of the subject compared to individuals that do not have the decrease in the level of PTHR1. In one embodiment, the difference is a decrease in the level of PTHR1 expression in the subject compared to the reference level and the decrease is an indication that the multiple myeloma is refractory multiple myeloma.

In one embodiment, the subject has previously been treated for multiple myeloma or has previously been diagnosed with multiple myeloma. In one embodiment, the reference level is the level of PTHR1 expression in a comparable sample from one or more healthy individuals. In one embodiment, the detecting is by amplifying a fragment of the PTHR1 mRNA. In one embodiment, the amplifying is by polymerase chain reaction (PCR) or RT-PCR. In one embodiment, the amplifying employs a detectably-labeled primer or probe.

In one embodiment, the detecting is by measuring the presence, absence, or amount of a PTHR1 protein in the test sample from the subject. In one embodiment, the measuring uses an antibody that specifically binds to the PTHR1 protein. In one embodiment, the measuring is by an ELISA assay, a western blot assay, or an immunohistochemical assay. In one embodiment, the test sample is a blood, serum, or biopsy sample. In one embodiment, the subject is a human patient having or suspected of having multiple myeloma, refractory multiple myeloma, or has relapsed to having multiple myeloma.

In one aspect, a method is provided for treating multiple myeloma in a patient, in need thereof, by administering to the patient an effective amount of one or more PTHR1 activators selected from PTH or PTH analogs or any combination thereof. In one embodiment, the method further includes administering to the patient an effective amount of one or more proteasome inhibitors selected from Bortezomib, Disulfuram, Salinosporamide A, Carfilzomib, CEP-18770, or MLN9708, or any combination thereof.

In one embodiment, the method further includes determining whether the patient will be a candidate for therapy, including one or more proteasome inhibitors, PTH, or PTH analogs, or any combination thereof, prior to the administering, wherein an increase in a level of PTHR1 expression in a test sample from the patient compared to a reference level indicates that the patient is a candidate for the foregoing therapies. In one embodiment, the reference level is the level of PTHR1 expression in a comparable sample from one or more healthy individuals.

### BRIEF DESCRIPTION OF THE FIGURES

FIG. 1 is a graph showing 5TGM1 myeloma cell proliferation in the presence or absence of increasing concentrations of bortezomib (velcade).

FIG. 2 is a graph showing 5TGM1 myeloma cell proliferation in the presence or absence of increasing concentrations of parathyroid hormone (PTH).

FIG. 3 is a graph showing 5TGM1 myeloma cell proliferation in the presence or absence of increasing concentrations of {[TYR³⁴]bPTH-(7-34)} (anti-PTH).

FIG. 4 is a graph showing 5TGM1 myeloma cell proliferation in the presence of bortezomib alone, PTH alone, and the combination of bortezomib and PTH.

FIG. 5 is a graph showing 5TGM1 myeloma cell proliferation in the presence of bortezomib alone, anti-PTH alone, and the combination of bortezomib and anti-PTH.

FIG. 6 is a graph showing the overall survival of mice treated with bortezomib alone, PTH alone, the combination of bortezomib and PTH, and the combination of bortezomib and anti-PTH.

FIG. 7 is a graph showing serum IgG concentrations of mice treated with bortezomib alone, PTH alone, the combination of bortezomib and PTH, and the combination of bortezomib and anti-PTH.

FIG. 8 is a graph showing the overall survival of mice treated with carfilzomib alone, anti-PTH alone, the combination of bortezomib and anti-PTH, and the combination of carfilzomib and anti-PTH.

FIGS. 9A and 9B are graphs showing the event free and overall survival of patients with MM, respectively. The graphs demonstrate that elevated PTHR1 levels are linked to an increased survival prognosis.

### DETAILED DESCRIPTION

The definitions of certain terms as used in this specification are provided below. Unless defined otherwise, all technical and scientific terms used herein generally have the same meaning as commonly understood by one of ordinary skill in the art to which this invention belongs.

As used in this specification and the appended claims, the singular forms “a”, “an” and “the” include plural referents unless the content clearly dictates otherwise. For example, reference to “a nucleic acid” includes a combination of two or more nucleic acids, and the like.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the enumerated value.

As used herein, the “administration” of an agent or drug to a subject or subject includes any route of introducing or delivering to a subject a compound to perform its intended function. Administration can be carried out by any suitable route, including orally, intranasally, parenterally (intravenously, intramuscularly, intraperitoneally, or subcutaneously), rectally, or topically. Administration includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

As used herein, the terms “amplification” or “amplify” mean one or more methods known in the art for copying a target nucleic acid, e.g., PTHR1 mRNA, thereby increasing the number of copies of a selected nucleic acid sequence. Amplification may be exponential or linear. A target nucleic acid may be either DNA or RNA. The sequences amplified in this manner form an “amplicon.” While the exemplary methods described hereinafter relate to amplification using the polymerase chain reaction (PCR), numerous other methods are known in the art for amplification of nucleic acids (e.g., isothermal methods, rolling circle methods, etc.). The skilled artisan will understand that these other methods may be used either in place of, or together with, PCR methods. See, e.g., Saiki, “Amplification of Genomic DNA” in PCR Protocols, Innis et al., Eds., Academic Press, San Diego, Calif. 1990, pp. 13-20; Wharam et al., Nucleic Acids Res., 2001, 29(11):E54-E54; Hafner et al., Biotechniques 2001, 30(4):852-6, 858, 860; Zhong et al., Biotechniques, 2001, 30(4):852-6, 858, 860.

As used herein the terms “analog” or “analogs”, in the context of a protein or polypeptide analog, or a PTH analog, refer to a variant of the protein or polypeptide that that has at least 40% sequence identity to the full-length, native, or natural protein or polypeptide. An analog may include a higher level of sequence identity, for example, at least 50%, at least 60%, at least 70%, at least 80%, at least 90%, at least 95%, at least 96%, at least 97%, at least 98% or at least 99% sequence identity. Analogs may include, but are not limited to, for example, proteins or polypeptides that are truncated, post-translationally modified, synthetically modified, mutated, labeled, denatured, degraded, derivatized, or proteins or polypeptides that have amino acid substitutions or deletions at one or more specific or random positions. In some embodiments, an analog may exhibit the same or different biological activity, such as agonist or antagonist activity, or an increase or reduction in biological activity compared to the native polypeptide or protein.

As noted above, an analog may also include fragments of a protein or polypeptide. Fragments lack 1, 2, 3, 4, 5, or more amino acids from any part of the sequence compared to the full-length or native polypeptide or protein. Thus, fragments include N-truncated and C-truncated polypeptides and proteins, but are not so limited. In some embodiments, the fragment has at least 20%, at least 30%, at least 40%, at least 50%, at least 60%, at least 70%, at least 80%, at least 90%, at least 95%, at least 96%, at least 97%, at least 98% or at least 99% of the total number of amino acids of the full-length or native polypeptide or protein. Fragments, like other analogs, have at least 40% sequence identity but may have any of the higher levels of sequence identity described herein. Such fragments, e.g., PTH fragments, and/or analogs, may be produced via recombinant DNA techniques, and isolated and/or purified naturally or synthetically using purification techniques well known in the art, e.g., affinity chromatography.

“Sequence identity” is defined herein with reference the Blast 2 algorithm, which is available at the NCBI (http://www.ncbi.nlm.nih.gov/BLAST), using default parameters. References pertaining to this algorithm include: those found at http://www.ncbi.nlm.nih.gov/BLAST/blast_references.html; Altschul, et al. “Basic local alignment search tool.” J. Mol. Biol. 215: 403-410 (1990); Gish, W. & States, D. J. “Identification of protein coding regions by database similarity search.” Nature Genet. 3: 266-272 (1993); Madden et al. “Applications of network BLAST server” Meth. Enzymol. 266: 131-141 (1996); Altschul et al. “Gapped BLAST and PSI-BLAST: a new generation of protein database search programs.” Nucleic Acids Res. 25: 3389-3402 (1997); and Zhang, J. & Madden, T. L. “PowerBLAST: A new network BLAST application for interactive or automated sequence analysis and annotation.” Genome Res. 7: 649-656 (1997). Accordingly, the peptide sequences from different species can be aligned, using standard computer programs like BLAST, to inform further variation domains that preserve their essential function.

As used herein the term “antibody” refers to an immunoglobulin and any antigen-binding portion of an immunoglobulin, e.g., IgG, IgD, IgA, IgM and IgE, or a polypeptide that contains an antigen binding site, which specifically binds or “immunoreacts with” an antigen. Antibodies can comprise at least one heavy (H) chain and at least one light (L) chain inter-connected by at least one disulfide bond. The term “V_{H}” refers to a heavy chain variable region of an antibody. The term “V_{L}” refers to a light chain variable region of an antibody. In exemplary embodiments, the term “antibody” specifically covers monoclonal and polyclonal antibodies. A “polyclonal antibody” refers to an antibody which has been derived from the sera of animals immunized with an antigen or antigens. A “monoclonal antibody” refers to an antibody produced by a single clone of hybridoma cells.

The term “clinical factors” as used herein, refers to any data that a medical practitioner may consider in determining a diagnosis or prognosis of disease. Such factors include, but are not limited to, the patient's medical history, a physical examination of the patient, complete blood count, etc.

The term “comparable” or “corresponding” in the context of comparing two or more samples, means that the same type of sample, e.g., tissue is used in the comparison. For example, an expression level of PTHR1 mRNA or protein in a tissue or biopsy sample can be compared to an expression level of PTHR1 in another whole blood sample. In some embodiments, comparable samples may be obtained from the same individual at different times. In other embodiments, comparable samples may be obtained from different individuals, e.g., a patient and a healthy individual. In general, comparable samples are normalized by a common factor. For example, body fluid samples are typically normalized by volume body fluid and cell-containing samples are normalized by protein content or cell count.

The terms “determining,” “measuring,” “assessing,” and “assaying” are used interchangeably and include both quantitative and qualitative determinations. These terms refer to any form of measurement, and include determining if a characteristic, trait, or feature is present or not. Assessing may be relative or absolute. “Assessing the presence of” includes determining the amount of something present, as well as determining whether it is present or absent.

As used herein, the term “diagnosis” means detecting a disease or disorder or determining the stage or degree of a disease or disorder. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease, e.g. there may be differential diagnoses that can be inferred from a diagnostic factor or symptom. Likewise, there may be instances where a factor or symptom that is indicative of a particular disease is present in an individual that does not have the particular disease. The term “diagnosis” also encompasses determining the therapeutic effect of a drug therapy, or predicting the pattern of response to a drug therapy. The diagnostic methods may be used independently, or in combination with other diagnosing and/or staging methods known in the medical arts for a particular disease or disorder, e.g., MM.

As used herein, the phrase “difference of the level” refers to differences in the quantity of a particular marker, such as a biomarker protein or nucleic acid, in a sample as compared to a control or reference level. For example, the quantity of particular protein or nucleic acid may be present at an elevated amount or at a decreased amount in samples of patients with a disease compared to a reference level. In one embodiment, a “difference of a level” may be a difference between the level of biomarker present in a sample as compared to a control of at least about 1%, at least about 2%, at least about 3%, at least about 5%, at least about 10%, at least about 15%, at least about 20%, at least about 25%, at least about 30%, at least about 35%, at least about 40%, at least about 50%, at least about 60%, at least about 75%, at least about 80% or more. In one embodiment, a “difference of a level” may be a statistically significant difference between the level of the biomarker present in a sample as compared to a control. For example, a difference may be statistically significant if the measured level of the biomarker falls outside of about 1.0 standard deviations, about 1.5 standard deviations, about 2.0 standard deviations, or about 2.5 stand deviations of the mean of any control or reference group.

As used herein, the term “effective amount” or “pharmaceutically effective amount” or “therapeutically effective amount” of a composition, is a quantity sufficient to achieve a desired therapeutic and/or prophylactic effect, e.g., an amount which results in the prevention of, or a decrease in, the symptoms associated with a disease that is being treated. The amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds and/or treatments.

The term “elevated levels” or “higher levels” as used herein refers to levels of a biomarker protein or nucleic acid that are higher than what would normally be observed in a comparable sample from control or normal subjects, e.g., a reference value. In some embodiments, “control levels”, e.g., normal levels, refer to a range of biomarker protein or nucleic acid levels that would normally be expected to be observed in a sample from a mammal that does not have a disease. A control level may be used as a reference level for comparative purposes. “Elevated levels” refer to biomarker protein or nucleic acid levels that are above the range of control levels. The ranges accepted as “elevated levels” or “control levels” are dependent on a number of factors. For example, one laboratory may routinely determine the level of biomarker protein or nucleic acid in a sample that are different than the level obtained for the same sample by another laboratory. Also, different assay methods may achieve different value ranges. Value ranges may also differ in various sample types, for example, different body fluids or by different treatments of the sample. One of ordinary skill in the art is capable of considering the relevant factors and establishing appropriate reference ranges for “control values” and “elevated values” of the present disclosure. For example, a series of samples from control subjects and subjects diagnosed with cancer can be used to establish ranges that are “normal” or “control” levels and ranges that are “elevated” or “higher” than the control range.

Similarly, “reduced levels” or “lower levels” as used herein refer to levels of a biomarker protein or nucleic acid that are lower than what would normally be observed in a comparable sample from control or normal subjects, e.g., a reference value. In some embodiments, “control levels”, e.g., normal levels, refer to a range of biomarker protein or nucleic acid levels that would be normally be expected to be observed in a mammal that does not have a disease and “reduced levels” refer to biomarker protein or nucleic acid levels that are below the range of control levels.

The term “enzyme linked immunosorbent assay” or “ELISA” as used herein refers to an antibody-based assay in which detection of the antigen of interest is accomplished via an enzymatic reaction producing a detectable signal. An ELISA can be run as a competitive or non-competitive format. ELISA also includes a 2-site or “sandwich” assay in which two antibodies to the antigen are used, one antibody to capture the antigen and one labeled with an enzyme or other detectable label to detect captured antibody-antigen complex. In a typical 2-site ELISA, the antigen has at least one epitope to which unlabeled antibody and an enzyme-linked antibody can bind with high affinity. An antigen can thus be affinity captured and detected using an enzyme-linked antibody. Typical enzymes of choice include alkaline phosphatase or horseradish peroxidase, both of which generate a detectable product when contacted by appropriate substrates.

As used herein, the terms “gene expression” or “expression” refer to the process of converting genetic information encoded in a gene into RNA, e.g., mRNA, rRNA, tRNA, or snRNA, through transcription of the gene, e.g., via the enzymatic action of an RNA polymerase, and for protein encoding genes, into protein through translation of mRNA. Gene expression can be regulated at many stages in the process. “Up-regulation” or “activation” refers to regulation that increases the production of gene expression products, e.g., RNA or protein, while “down-regulation” or “repression” or “knock-down” refers to regulation that decrease production. Molecules, e.g., transcription factors that are involved in up-regulation or down-regulation are often called “activators” and “repressors,” respectively.

As used herein, the term “introduce” refers to the incorporation of a nucleic acid into a eukaryotic or prokaryotic cell where the nucleic acid may be incorporated into the genome of the cell, e.g., chromosome, plasmid, plastid, or mitochondrial DNA, converted into an autonomous replicon, or transiently expressed, e.g., infected mRNA. The term includes such nucleic acid introduction means as transfection, transformation, and transduction.

As used herein, “microarray” or “gene expression array” or “array” or “tissue microarray” refers to an arrangement of a collection of nucleic acids, e.g., nucleotide sequences in a centralized location. Arrays can be on a solid substrate, such as a glass slide, or on a semi-solid substrate, such as nitrocellulose membrane. The nucleotide sequences can be DNA, RNA, or any combination or permutations thereof. The nucleotide sequences can also be partial sequences or fragments from a gene, primers, whole gene sequences, non-coding sequences, coding sequences, published sequences, known sequences, or novel sequences. Tissue microarrays are well known in the art and can be performed as described. See e.g., Camp, R. L., et al., J Clin Oncol, 26, 5630-5637 (2008).

As used herein, “nucleic acid” refers broadly to segments of a chromosome, segments or portions of DNA, cDNA, and/or RNA. Nucleic acid may be derived or obtained from an originally isolated nucleic acid sample from any source, e.g., isolated from, purified from, amplified from, cloned from, or reverse transcribed from sample DNA or RNA.

As used herein, the term “nucleic acid fragment” refers to a sequence of nucleotide residues which are at least about 5 nucleotides, at least about 7 nucleotides, at least about 9 nucleotides, at least about 11, nucleotides, or at least about 17, nucleotides. The fragment is typically less than about 300 nucleotides, less than about 100 nucleotides, less than about 75 nucleotides less than about 50 nucleotides, or less than about 30 nucleotides. In certain embodiments, the nucleic acid fragments can be used in polymerase chain reaction (PCR), or various hybridization procedures to identify or amplify identical or related DNA or RNA molecules.

As used herein, the term “oligonucleotide” refers to a short polymer composed of deoxyribonucleotides, ribonucleotides or any combination thereof. Oligonucleotides are generally between about 10 and about 100 nucleotides in length. Oligonucleotides are typically 15 to 70 nucleotides long, with 20 to 26 nucleotides being the most common. An oligonucleotide may be used as a primer or as a probe. An oligonucleotide is “specific” for a nucleic acid if the oligonucleotide has at least 50% sequence identity with a portion of the nucleic acid when the oligonucleotide and the nucleic acid are aligned. An oligonucleotide that is specific for a nucleic acid is one that, under the appropriate hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, or at least 95% sequence identity.

As used herein, the term “overall survival” or “OS” is used to refer to time in years from treatment to death from any cause. The calculation of this measure may vary depending on the definition of events to be either censored or not considered.

As used herein, the term “p-value” or “p” refers to a measure of probability that a difference between groups happened by chance. For example, a difference between two groups having a p-value of 0.01 (or p=0.01) means that there is a 1 in 100 chance the result occurred by chance. Suitable p-values include, but are not limited to, 0.1, 0.05, 0.025, 0.02, 0.01, 0.005, 0.001, and 0.0001.

As used herein, the terms “polypeptide,” “peptide,” and “protein” are used interchangeably to refer to a polymer of amino acid residues. The terms apply to amino acid polymers in which one or more amino acid residues is an artificial chemical analog of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms polypeptide, peptide, and protein are also inclusive of modifications including, but not limited to, glycosylation, lipid attachment, sulfation, carboxylation, hydroxylation, ADP-ribosylation, and addition of other complex polysaccharides.

As used herein, a “primer” for amplification is an oligonucleotide that specifically anneals to a target or marker nucleotide sequence. The 3′ nucleotide of the primer should be identical to the target or marker sequence at a corresponding nucleotide position for optimal primer extension by a polymerase. As used herein, a “forward primer” is a primer that anneals to the anti-sense strand of double stranded DNA (dsDNA). A “reverse primer” anneals to the sense-strand of dsDNA.

The term “prognosis” as used herein refers to a prediction of the probable course and outcome of a clinical condition or disease. A prognosis is usually made by evaluating factors or symptoms of a disease that are indicative of a favorable or unfavorable course or outcome of the disease. The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. The terms “favorable prognosis” and “positive prognosis,” or “unfavorable prognosis” and “negative prognosis” as used herein are relative terms for the prediction of the probable course and/or likely outcome of a condition or a disease. A favorable or positive prognosis predicts a better outcome for a condition than an unfavorable or negative prognosis. In a general sense, a “favorable prognosis” is an outcome that is relatively better than many other possible prognoses that could be associated with a particular condition, whereas an unfavorable prognosis predicts an outcome that is relatively worse than many other possible prognoses that could be associated with a particular condition. Typical examples of a favorable or positive prognosis include a better than average remission rate, a lower propensity for metastasis, a longer than expected life expectancy, differentiation of a benign process from a cancerous process, and the like.

As used herein, the term “reference level” refers to a level of a substance which may be of interest for comparative purposes. In one embodiment, a reference level may be the expression level of a protein or nucleic acid expressed as an average of the level of the expression level of a protein or nucleic acid from samples taken from a control population of healthy (disease-free) subjects. In another embodiment, the reference level may be the level in the same subject at a different time, e.g., before the present assay, such as the level determined prior to the subject developing the disease or prior to initiating therapy. In general, samples are normalized by a common factor. For example, body fluid samples are normalized by volume body fluid and cell-containing samples are normalized by protein content or cell count.

As used herein, the term “sample” or “test sample” refers to any liquid or solid material containing nucleic acids or proteins. In suitable embodiments, a test sample is obtained from a biological source, e.g., a “biological sample”, such as cells in culture or a tissue sample from an animal, most preferably, a human. In an exemplary embodiment, the sample is a tumor sample.

As used herein, the term “subject” refers to a mammal, such as a human, but can also be another animal such as a domestic animal, e.g., a dog, cat, or the like, a farm animal, e.g., a cow, a sheep, a pig, a horse, or the like, or a laboratory animal, e.g., a monkey, a rat, a mouse, a rabbit, a guinea pig, or the like. The term “patient” refers to a “subject” who is, or is suspected to be, afflicted with MM.

As used herein, the term “simultaneous” therapeutic use refers to the administration of at least two active ingredients by the same route and at the same time or at substantially the same time.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, “target nucleic acid” refers to segments of a chromosome, a complete gene with or without intergenic sequence, segments or portions a gene with or without intergenic sequence, or sequence of nucleic acids to which probes or primers are designed. Target nucleic acids may be derived from genomic DNA, cDNA, or RNA. As used herein, target nucleic acid may be native DNA or a PCR-amplified product. In one embodiment, the target nucleic acid is a fragment of a chromosome to be analyzed for methylation, e.g., a promoter region of a gene. In some embodiments, the target nucleic acid is a segment of the PTHR1 mRNA.

As used herein, the terms “treating” or “treatment” or “alleviation” refers to both therapeutic treatment and prophylactic or preventative measures, wherein the object is to prevent or slow down (lessen) the targeted pathologic condition or disorder. A subject is successfully “treated” for a disorder if, after receiving a therapeutic agent according to the methods of the present disclosure, the subject shows observable and/or measurable reduction in or absence of one or more signs and symptoms of a particular disease or condition.

### Overview

The present disclosure relates to the diagnosis, prognosis, and treatment of multiple myeloma (MM). MM is an aggressive cancer in which drug-resistant disease remains a significant clinical problem. Accordingly, the present disclosure relates to the diagnosis of MM oncogenesis, including the prognostic determination thereof. The present disclosure further includes methods for determining and predicting MM oncogenic phenotypes, such as drug-resistant or refractory MM. Methods are described for the quantification of mRNA or protein levels of a parathyroid hormone (PTH) receptor biomarker in order to assist an oncologist in determining a suitable treatment regime for patients afflicted with MM or that have relapsed from a prior state of remission.

It has been discovered that over-expression of PTH correlates with the oncogenic capacity of MM. PTH, also known as parathyrin, is synthesized and secreted by the parathyroid glands as an 84 amino acid polypeptide, which functions to increase serum calcium concentration. Reductions in extracellular calcium levels are detected by calcium sensing receptors on parathyroid chief cells and, in response, promote the release of PTH, which operates to increase bone resorption, thereby restoring basal calcium levels.

Full-length PTH (1-84) is the predominant form of secreted PTH. However, recent analyses reveal that amino-terminal truncations of PTH are similarly secreted by the parathyroid glands and generated by peripheral metabolism. Such PTH fragments and/or their synthetic analogs are inert PTH receptor substrates because, despite receptor binding, they fail to induce activation of adenylyl cyclase and phospholipase C. In fact, PTH fragments lacking part of the amino-terminus behave as competitive PTH antagonists. See Sneddon et al., Activation-independent Parathyroid Hormone Receptor Internalization Is Regulated by NHERF1 (EBP50). J Biol. Chem. 278 (44): 43787-96 (2003).

As described herein, PTH involvement in MM disease progression was confirmed via gene expression analyses, which indicated that the expression of one specific PTH receptor, PTHR1, significantly impacts overall and event free survival of patients receiving MM treatment. Such treatments include, but are not limited to, proteasome inhibitor treatments, PTH augmentation therapies, and/or combinations thereof. Thus, the PTH/PTHR1 system appears to be directly involved in the regulation of antineoplastic biofeedback pathways.

In one aspect, the present disclosure provides for the prognostic determination of whether a patient with MM will be responsive to particular treatments or therapies based on an elevated or reduced expression of a PTH receptor. In one embodiment, the PTH receptor is parathyroid hormone/parathyroid hormone-related peptide receptor (PTHR1), which is also known as parathyroid hormone 1 receptor (PTH1R). PTHR1 is encoded by the PTH1R gene, and primarily functions as a receptor for PTH and for parathyroid hormone-related protein (PTHrP), also known as parathyroid hormone-like hormone (PTHLH). A determination of reduced PTHR1 expression, compared to a reference level, may indicate that the MM is resistant to certain chemotherapeutic agents, e.g., the MM is refractory MM. In another embodiment, a determination of increased PTHR1 expression, compared to a reference level, indicates that the MM is sensitive to chemotherapeutic agents, such as, but not limited to, proteasome inhibitors and/or PTHR1 activators, e.g., PTH, PTH analogs, and the like.

The present disclosure also includes methods for determining and treating MM oncogenesis, including the identification and treatment of refractory MM and/or MM, respectively. Methods for treating MM include administering effective amounts of one or more PTHR1 activators. The methods of the present disclosure further provide treatment regimes that may be selected based upon the diagnosis or prognosis of MM. In one embodiment, a treatment regime is selected based upon elevated expression levels of PTHR1.

The methods further include individual or combination therapies employing PTHR1 activators in the presence or absence of additional chemotherapeutic agents. Individual or combination therapies are beneficial when it is determined, prior to treatment, that particular therapies, e.g., proteasome inhibitor or PTH therapy, will increase a patients overall survival. Such determinations are based on MM biomarker levels, expression, or activation thereof, e.g., PTHR1 activation.

In suitable embodiments, it can be determined that a MM patient will be responsive to proteasome inhibitor and/or PTH therapy when the results indicate elevated levels of one or more MM biomarkers, e.g., PTHR1 mRNA or a PTHR1 protein, in a sample. In one embodiment, elevated or increased levels of PTHR1 mRNA or a PTHR1 receptor protein, when compared to a reference level, is prognostic for an increased overall survival of a MM patient when the patient is also treated with proteasome inhibitors, e.g., bortezomib, disulfuram, salinosporamide A, carfilzomib, CEP-18770, or MLN9708, or any combination thereof. Furthermore, decreased levels of PTHR1 mRNA or a PTHR1 protein, when compared to a reference level, may be prognostic for refractory MM. In one embodiment, decreased levels of PTHR1 mRNA or a PTHR1 protein, when compared to a reference level, is prognostic for a decreased overall survival of a MM patient.

In addition to PTHR1 and/or PTH biomarkers, supplementary diagnostic markers may be combined with a PTHR1 expression profile to construct models for predicting the presence or absence or stage of a disease, e.g., MM. For example, relevant clinical factors for diagnosing MM, or various phenotypes thereof, including susceptibility to certain treatment regimes, include, but are not limited to, the subject's medical history, a physical examination, complete blood count, and other biological or biochemical markers. Moreover, biomarkers relevant to MM prognosis may be combined with a patient's PTHR1 expression profile for diagnosis or prognosis, e.g., NEK2 mRNA or protein expression levels.

Nevertheless, the MM biomarkers described herein are capable of detecting the presence or absence of MM or a particular MM phenotype, e.g., refractory MM, without further assessment or determination. Such biomarkers can be quantitatively or qualitatively determined by empirically or relatively measuring levels thereof, respectively, in a sample from a subject. The samples include, but are not limited to, sputum, blood (or a fraction of blood such as plasma, serum, or particular cell fractions), lymph, mucus, tears, saliva, urine, semen, ascites fluid, whole blood, and biopsy samples of body tissue, e.g., bone or bone marrow. In one embodiment, the sample is a bone marrow core biopsy tissue sample from a patient suspected of having MM.

In this regard, by employing the present methods, results from a particular sample may reveal that that patient can be efficaciously treated with, for example, proteasome inhibitors and/or PTH, e.g., when mRNA or protein expression levels within a sample are elevated relative to a reference level. In one embodiment, the proteasome inhibitor is bortezomib, disulfuram, salinosporamide A, carfilzomib, CEP-18770 and/or MLN9708. In another embodiment, PTH therapy, including PTH stimulation, augmentation, and replacement therapy, is employed, and includes, administration of PTHR1 activators, PTH, and/or PTH analogs, and the like.

### Sample Collection and Preparation

The methods and compositions described herein may be used to detect nucleic acids associated with various genes using a biological sample obtained from an individual. The nucleic acid (DNA or RNA) may be isolated from the sample according to any methods well known to those of skill in the art. Biological samples may be obtained by standard procedures and may be used immediately or stored, under conditions appropriate for the type of biological sample, for later use.

Starting material for the detection assays is typically a clinical sample, which is suspected to contain the target nucleic acids, e.g., PTHR1 mRNA. An example of a clinical sample is bone marrow tissue. The nucleic acids may be separated from proteins and/or other constituents in the original sample. Any purification methods known in the art may be used in the context of the present invention. Nucleic acid sequences in the sample can successfully be amplified using in vitro amplification, such as PCR. Typically, any compounds that may inhibit polymerases are removed from the nucleic acids.

Methods of obtaining samples are well known to those of skill in the art and include, but are not limited to, aspirations, tissue sections, swabs, drawing of blood or other fluids, surgical or needle biopsies, and the like. The sample may be obtained from an individual or patient. The sample may contain cells, tissues, bone or fluid obtained from a patient suspected being afflicted with multiple myeloma. The sample may be a cell-containing liquid or a tissue. Samples may include, but are not limited to, biopsies, bone biopsies, bone marrow biopsies, blood, blood cells, bone marrow, fine needle biopsy samples, plasma, pleural fluid, saliva, serum, tissue or tissue homogenates, frozen or paraffin sections of tissue. Samples may also be processed, such as sectioning of tissues, fractionation, purification, or cellular organelle separation.

If necessary, the sample may be collected or concentrated by centrifugation and the like. The cells of the sample may be subjected to lysis, such as by treatments with enzymes, heat, surfactants, ultrasonication, or a combination thereof. The lysis treatment is performed in order to obtain a sufficient amount of nucleic acid derived from the cells in the sample to detect using polymerase chain reaction.

### Methods for Nucleic Acid Detection

The nucleic acid to be amplified may be from a biological sample such as a tissue sample or bone marrow sample, and the like. Various methods of extraction are suitable for isolating the DNA or RNA. Suitable methods include phenol and chloroform extraction. See Maniatis et al., Molecular Cloning, A Laboratory Manual, 2d, Cold Spring Harbor Laboratory Press, pp. 16-54 (1989). Numerous commercial kits also yield suitable DNA and RNA including, but not limited to, QIAamp™ mini blood kit, Agencourt Genfind™, Roche Cobas® Roche MagNA Pure® or phenol: chloroform extraction using Eppendorf Phase Lock Gels®, and the NucliSens extraction kit (Biomerieux, Marcy l'Etoile, France).

Nucleic acid extracted from cells or tissues can be amplified using nucleic acid amplification techniques well known in the art. By way of example, but not by way of limitation, these techniques can include polymerase chain reaction (PCR), reverse transcriptase polymerase chain reaction (RT-PCR), nested PCR, ligase chain reaction. See Abravaya, K., et al., Nucleic Acids Research, 23:675-682, (1995), branched DNA signal amplification, Urdea, M. S., et al., AIDS, 7 (suppl 2):S11-S14, (1993), amplifiable RNA reporters, Q-beta replication, transcription-based amplification, boomerang DNA amplification, strand displacement activation, cycling probe technology, isothermal nucleic acid sequence based amplification (NASBA). See Kievits, T. et al., J Virological Methods, 35:273-286, (1991), Invader Technology, or other sequence replication assays or signal amplification assays may also be used. Some of these methods of amplification are described briefly below and are well-known in the art.

Some methods employ reverse transcription of RNA to cDNA. The method of reverse transcription and amplification may be performed by previously published or recommended procedures. Various reverse transcriptases may be used, including, but not limited to, MMLV RT, RNase H mutants of MMLV RT such as Superscript and Superscript II (Life Technologies, GIBCO BRL, Gaithersburg, Md.), AMV RT, and thermostable reverse transcriptase from Thermus thermophilus. For example, one method which may be used to convert RNA to cDNA is the protocol adapted from the Superscript II Preamplification system (Life Technologies, GIBCO BRL, Gaithersburg, Md.; catalog no. 18089-011), as described by Rashtchian, A., PCR Methods Applic., 4:S83-S91, (1994).

In one embodiment, PCR is used to amplify a target sequence of interest, e.g., a PTHR1 sequence. PCR is a technique for making many copies of a specific template DNA sequence. The reaction consists of multiple amplification cycles and is initiated using a pair of primer sequences that hybridize to the 5′ and 3′ ends of the sequence to be copied. The amplification cycle includes an initial denaturation, and typically up to 50 cycles of annealing, strand elongation and strand separation (denaturation). In each cycle of the reaction, the DNA sequence between the primers is copied. Primers can bind to the copied DNA as well as the original template sequence, so the total number of copies increases exponentially with time. PCR can be performed as according to Whelan et al., J of Clin Micro, 33(3):556-561 (1995). Briefly, a PCR reaction mixture includes two specific primers, dNTPs, approximately 0.25 U of Taq polymerase, and 1×PCR Buffer.

The skilled artisan is capable of designing and preparing primers that are appropriate for amplifying a target or marker sequences. The length of the amplification primers depends on several factors including the nucleotide sequence identity and the temperature at which these nucleic acids are hybridized or used during in vitro nucleic acid amplification. The considerations necessary to determine a preferred length for an amplification primer of a particular sequence identity are well-known to a person of ordinary skill in the art. For example, the length of a short nucleic acid or oligonucleotide can relate to its hybridization specificity or selectivity. Exemplary primers for detecting PTHR1 mRNA may be designed based on the nucleotide sequence available at GenBank Accession No NP_{—}000307.

In some embodiments, the amplification may include a labeled primer or probe, thereby allowing detection of the amplification products corresponding to that primer or probe. In one embodiment, the amplification may include a multiplicity of labeled primers or probes; such primers may be distinguishably labeled, allowing the simultaneous detection of multiple amplification products. In suitable embodiments, a primer or probe is labeled with a fluorogenic reporter dye that emits a detectable signal. While an appropriate reporter dye may be a fluorescent dye, any reporter dye that can be attached to a detection reagent such as an oligonucleotide probe or primer is suitable for use in the invention. Such dyes include, but are not limited to, Acridine, AMCA, BODIPY, Cascade Blue, Cy2, Cy3, Cy5, Cy7, Edans, Eosin, Erythrosin, Fluorescein, 6-Fam, Tet, Joe, Hex, Oregon Green, Rhodamine, Rhodol Green, Tamra, Rox, and Texas Red.

In another embodiment, the detection reagent may be further labeled with a quencher dye such as Tamra, Dabcyl, or Black Hole Quencher® (“BHQ”), especially when the reagent is used as a self-quenching probe such as a TaqMan°, See U.S. Pat. Nos. 5,210,015 and 5,538,848, or Molecular Beacon probe, See U.S. Pat. Nos. 5,118,801 and 5,312,728, or other stemless or linear beacon probes. See Livak et al., PCR Method Appl., 4:357-362 (1995); Tyagi et al, Nature Biotechnology, 14:303-308 (1996); Nazarenko et al., Nucl. Acids Res., 25:2516-2521 (1997); and U.S. Pat. Nos. 5,866,336 and 6,117,635.

Nucleic acids may be amplified prior to detection or may be detected directly during an amplification step, e.g., “real-time” methods. In some embodiments, the target sequence is amplified using a labeled primer such that the resulting amplicon is detectably labeled. In some embodiments, the primer is fluorescently labeled. In some embodiments, the target sequence is amplified and the resulting amplicon is detected by electrophoresis.

The level of gene expression can be determined by assessing the amount of PTHR1 mRNA in a test sample. Methods of measuring mRNA in samples are known in the art. To measure mRNA levels, the cells in the samples can be lysed and the levels of mRNA in the lysates or in RNA purified or semi-purified from lysates can be measured by any variety of methods familiar to those in the art. Such methods include, without limitation, hybridization assays using detectably labeled DNA or RNA probes, e.g., northern blotting, or quantitative or semi-quantitative RT-PCR methodologies using appropriate oligonucleotide primers. Alternatively, quantitative or semi-quantitative in situ hybridization assays can be carried out using, for example, tissue sections, or unlysed cell suspensions, and detectably labeled, e.g., fluorescent, or enzyme-labeled, DNA or RNA probes. Additional methods for quantifying mRNA include RNA protection assay (“RPA”), cDNA and oligonucleotide microarrays, representation difference analysis (“RDA”), differential display, EST sequence analysis, serial analysis of gene expression (“SAGE”), and multiplex ligation-mediated amplification with the Luminex FlexMAP (“LMF”). See Peck et al., Genome Biol., 7(7):R61 (2006).

Amplification can also be monitored using “real-time” methods. Real time PCR allows for the detection and quantitation of a nucleic acid target. Typically, this approach to quantitative PCR utilizes a fluorescent dye, which may be a double-strand specific dye, such as SYBR Green® I. Alternatively, other fluorescent dyes, e.g., FAM or HEX, may be conjugated to an oligonucleotide probe or a primer. Various instruments capable of performing real time PCR are known in the art and include, for example, ABI Prism® 7900 (Applied Biosystems) and LightCycler® systems (Roche). The fluorescent signal generated at each cycle of PCR is proportional to the amount of PCR product. A plot of fluorescence versus cycle number is used to describe the kinetics of amplification and a fluorescence threshold level is used to define a fractional cycle number related to initial template concentration. When amplification is performed and detected on an instrument capable of reading fluorescence during thermal cycling, the intended PCR product from non-specific PCR products can be differentiated using melting analysis. By measuring the change in fluorescence while gradually increasing the temperature of the reaction subsequent to amplification and signal generation it may be possible to determine the Tₘ of the intended product(s) as well as that of the nonspecific product.

The methods may include amplifying multiple nucleic acids in sample, also known as “multiplex detection” or “multiplexing.” As used herein, the term “multiplex PCR” refers to PCR, which involves adding more than one set of PCR primers to the reaction in order to detect and quantify multiple nucleic acids, including nucleic acids from one or more target gene markers. Furthermore, multiplexing with an internal control, e.g., 18s rRNA, GADPH, or β-actin) provides a control for the PCR without reaction.

In one embodiment, the methods include measuring the level of PTHR1 mRNA transcript. Microarrays are an array of distinct polynucleotides or oligonucleotides synthesized on a substrate, such as paper, nylon or other type of membrane, filter, chip, glass slide, or any other suitable solid support. In other embodiments, the microarray is composed of a large number of unique, single-stranded nucleic acid sequences, usually either synthetic antisense oligonucleotides or fragments of cDNAs, fixed to a solid support. Polynucleotides used in the microarray may be oligonucleotides or fragments that are specific to a gene or genes of interest, e.g., PTHR1.

Fluorescently-labeled single strand (or “first strand”) cDNA probes can be synthesized from total RNA or mRNA by first isolating RNA from the sample of cells to be tested and cells of a control or reference sample. Typically, the two cDNA samples are labeled with different fluorescent dyes, e.g. green Cy3 and red Cy5. The two labeled cDNA samples are mixed and hybridized to the microarray, and the slide is scanned. In the resulting image, the green Cy3 and red Cy5 signals are overlaid—yellow spots indicate equal intensity for the dyes. With the use of image analysis software, signal intensities are determined for each dye at each element of the array, and the logarithm of the ratio of Cy5 intensity to Cy3 intensity is calculated (center). Positive log(Cy5/Cy3) ratios indicate relative excess of the transcript in the Cy5-labeled sample, and negative log(Cy5/Cy3) ratios indicate relative excess of the transcript in the Cy3-labeled sample. Values near zero indicate equal abundance in the two samples. In one embodiment, tissue microarray analysis (“TMA”), can be employed for PTHR1 mRNA detection when the sample is a tissue sample or a bone marrow sample. See Camp, R. L., et al., J Clin Oncol, 26, 5630-5307 (2008).

### Methods for Conducting Protein Assays

In one embodiment, the methods provide for detection of PTHR1 protein levels. The presence of PTHR1 can be measured by immunoassay, using antibodies specific for PTHR1 protein. A lack of antibody binding would indicate the absence of PTHR1 protein molecules, or a decreased level thereof, and may suggest that the patient would not be responsive to proteasome inhibitor therapy or PTH therapy as described herein. Exemplary PTHR1 antibodies are commercially available from Abcam (Cambridge, UK) and Abnova (Walnut, Calif., USA).

PTHR1 antibodies may be obtained in a number of ways which will be readily apparent to those skilled in the art. The protein can be produced in a recombinant system using the nucleotide sequence of PTHR1 (GenBank Accession No. NP_{—}000307). The recombinant protein can be injected into an animal as an immunogen to elicit polyclonal antibody production. The resultant polyclonal antisera may be used directly or may be purified by, for example, affinity absorption using recombinantly produced PTHR1 coupled to an insoluble support.

PTHR1 proteins can also be detected by immunohistochemistry, immunofluorescence, ELISPOT, ELISA, or RIA. The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986). Immunoassays are binding assays involving interactions between antibodies and antigen. Many types and formats of immunoassays are known and all are suitable for detecting the disclosed biomarker, e.g., PTHR1. Examples of immunoassays are enzyme linked immunosorbent assays (“ELISAs”), enzyme linked immunospot assay (ELISPOT), radioimmunoassays (“RIA”), radioimmune precipitation assays (RIPA), immunobead capture assays, western blotting, dot blotting, gel-shift assays, flow cytometry, immunohistochemistry, fluorescence microscopy, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label. See, e.g., U.S. Pat. Nos. 3,817,837; 3,850,752; 3,939,350; 3,996,345; 4,277,437; 4,275,149 and 4,366,241.

In one embodiment, the level of PTHR1 protein or mRNA expression, in a sample, can be compared with levels observed in a control sample. A control may be any sample of cells or tissue where MM is absent. For example, a control sample may be non-cancerous cells, including, but not limited to normal human fibroblasts, human umbilical endothelial cells (HUVECs), or mesenchymal stem cells. A control sample may further include any tumor cell type that is not a MM tumor cell including, for example, HEK293, HeLa, HCT116, MCF7, 501MEL, LNCaP, PC3, BT-20, SK-BR-3, and/or SK-OV-3. Also, any other pediatric tumor type where MM is absent would be an appropriate control sample. These could include HOS, OST, SAOS, MG-63, U2OS, RD, TTC442, CCL-136, HR, JR, RH28, RH30, Birch, CHLA 20, CHP 126, and/or CHLA 90.

A control sample may also be cancerous cells derived from MM patients. For example, a control sample may be myeloma cells, which include, but are not limited to, 5TGM1, 5T33, RPMI 8226, ARP1, XG 1, XG 2, XG 3, XG 4, XG 5, XG 6, U266, RPM1 8226, LP1, L363, OPM2, and/or NCLH929 cells, etc. See, e.g., Lombardi et al., Molecular characterization of human multiple myeloma cell lines by integrative genomics: insights into the biology of the disease. Genes Chromosomes Cancer. 46 (3): 226-38 (2007).

### Diagnosis of Disease States

In one aspect of the present disclosure, the information obtained from comparative PTHR1 expression profiles are employed to determine a patient's MM diagnosis, MM prognosis, or a suitable treatment regime therefor. If PTHR1 is expressed at a higher level in the patient's sample compared to a reference level then it is likely that the sample is from a patient that will be responsive to proteasome inhibitor therapy or to PTH therapy. On the other hand, if PTHR1 is expressed at a lower or decreased level in the patient's sample compared to a reference level then it is likely that the sample is from a patient harboring refractory MM, which may not be responsive to proteasome inhibitor therapy or to PTH therapy.

In addition, an increase or decrease in PTHR1 expression levels may be used in conjunction with other clinical factors to determine a patients prognosis. Moreover, PTHR1 expression can be employed for determining whether a subject is a candidate for treatment. In one embodiment, if the subject is a patient with MM and decreased levels of PTHR1 expression, compared to controls, e.g., reference levels, then the patient is not a likely candidate for treatment. In another embodiment, if the subject is a patient with MM and elevated levels of PTHR1 expression, compared to a reference level, then the patient is a candidate for treatment. Such treatment includes proteasome inhibitors, PTH therapy, including PTH analog therapy, and various other chemotherapeutic therapies as described herein.

In one aspect, if it is determined that the patient is not a suitable candidate for MM-related therapies, e.g., proteasome inhibitors and PTH therapy, then the patient is at risk for decreased overall survival. Accordingly, if it is determined that the patient is a suitable candidate for the MM-related therapies, then the patient has an increased overall survival when suitable treatments are administered, e.g., proteasome inhibitors. As such, the present disclosure also provides for prognostic overall survival determination of a patient based on PTHR1 levels in a sample. In this regard, the present disclosure provides for determining the mortality risk of MM patients based at least partially on results of tests from a sample. In one embodiment, overall survival determination or mortality risk is based on PTHR1 expression levels in a sample. In suitable embodiments, the overall survival determination or mortality risk is based on PTHR1 expression levels in a sample when compared to a control sample.

### Compositions and Methods for MM Treatment

One aspect of the present disclosure identifies PTHR1 as a target for therapeutic intervention. Activating or increasing the expression of PTHR1 in cancerous cells can prevent or disrupt the ability of MM cells to propagate. As such, the present disclosure provides for compositions and methods that are useful for modulating the activation of PTHR1 to treat MM, including patients that have relapsed to MM. In one aspect, the present disclosure includes compositions, which include therapeutic agents and pharmaceutically acceptable carriers therefor.

In this regard, the present disclosure provides for one or more therapeutic agents, e.g., proteasome inhibitors or PTH, including analogs thereof, which are administered to a MM patient. The therapeutic agents can be administered to a patient prior to, during, or after other conventional chemotherapeutic treatments. In one embodiment, the therapeutic agents are administered to a patient subsequent to determining that the patient is a candidate for such treatment. In this respect, the therapeutic agents are administered to a subject, prior to, or in combination with, conventional chemotherapeutic treatments.

In one aspect, the therapeutic agents, alone or in combination, are administered to a patient in an effective amount, e.g., a therapeutically effective dose of a PTHR1 activator and/or proteasome inhibitor. A therapeutic dose may vary depending upon the type of therapeutic agent, route of administration, and dosage form. Dosage unit forms generally contain between from about 1 mg to about 500 mg of an active ingredient. The preferred composition or compositions is a formulation that exhibits a high therapeutic index. The therapeutic index is the dose ratio between toxic and therapeutic effects which can be expressed as the ratio between LD50 and ED50. The LD50 is the dose lethal to 50% of the population and the ED50 is the dose therapeutically effective in 50% of the population. The LD50 and ED50 are determined by standard pharmaceutical procedures in animal cell cultures or experimental animals.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation.

In the compositions for treating MM described herein, the therapeutically effective amount of the proteasome inhibitor and/or PTHR1 activator (the agent) can range from about 0.001 mg/kg to about 30 mg/kg body weight of the subject. In some embodiments, the therapeutically effective amount of the agent can range from about 0.05 mg/kg to about 30 mg/kg, from about 0.1 mg/kg to about 30 mg/kg, from about 1 mg/kg to about 25 mg/kg, from about 1 mg/kg to about 20 mg/kg, or from about 1 or 2 mg/kg to about 15 mg/kg.

In one embodiment, the therapeutic agents are proteasome inhibitors, such as, but not limited to, bortezomib, disulfuram, salinosporamide A, carfilzomib, CEP-18770, and/or MLN9708, or any combination thereof. In another embodiment, the therapeutic agents are PTHR1 activators, which include the full-length PTH polypeptide, a fragment of the PTH polypeptide, an amino-terminal fragment of the PTH polypeptide, or any analog thereof. In suitable embodiments, combination therapies are employed, which include one or more proteasome inhibitors and PTH polypeptide, fragments, or analogs thereof.

Along these lines, the in vivo administration of PTH polypeptides and/or analogs thereof, e.g., teriparatide—a recombinant PTH polypeptide composed of the first 34 amino acid, which has been employed for treatment of postmenopausal osteoporosis, can result in a rapid increase in bone-formation. Nevertheless, the increase is typically followed by increases in bone-resorption. Accordingly, precise treatment regimes, as disclosed herein, are required to maximize therapeutic efficacy during the “anabolic window”, e.g., a period of time when PTH treatment is optimally anabolic. See Gallacher et al., Impact of treatments for postmenopausal osteoporosis (Biphosphonates, Parathyroid Hormone, Strontium Ranelate, and Denosumab) on Bone Quality: A Systematic Review. Calcif Tissue Int. Abstract (2010). Such treatment regimes, for example, PTHR1 activators, can influence the ubiquitin proteasome pathway.

The ubiquitin proteasome pathway is an essential degradative system in myeloma cells, which can also regulate bone formation via inducing osteoblast differentiation. See, e.g., Tian et al., The role of the Wnt-signaling antagonist DKK1 in the development of osteolytic lesions in multiple myeloma. N Engl J Med. 349(26): 2483-94 (2003); Garrett et al., Selective inhibitors of the osteoblast proteasome stimulate bone formation in vivo and in vitro. J Clin Invest. 111(11): 1771-82 (2003). Moreover, the proteasome inhibitor, bortezomib, can inhibit myeloma progression in the myeloma SCID-hu model in vivo. See Pennisi et al., The proteasome inhibitor, bortezomib suppresses primary myeloma and stimulates bone formation in myelomatous and nonmyelomatous bones in vivo. Am J. Hematol. 2009; 84(1):6-14 (2009). In one embodiment, the proteasome inhibitors bortezomib and/or carfilzomib are administered to a MM patient. In another embodiment, bortezomib and/or carfilzomib are administered to a MM patient with elevated levels of PTHR1 expression.

In this regard, bortezomib can be administered at a concentration from about 0.001-30, 0.05-30, 0.1-30, 1-30, 1-25, 1-20, 1-15, or 1-10 mg/kg body weight of the patient. In one embodiment, bortezomib is administered at a concentration from about 1-10 mg/kg body weight of the patient. In another embodiment, bortezomib is administered at a concentration of about 5 mg/kg body weight of the patient. In a similar fashion, carfilzomib can be administered at a concentration from about 0.001-30, 0.05-30, 0.1-30, 1-30, 1-25, 1-20, 1-15, or 1-10 mg/kg body weight of the patient. In another embodiment, carfilzomib is administered at a concentration from about 1-10 mg/kg body weight of the patient. In one embodiment carfilzomib is administered at a concentration of about 5 mg/kg body weight of the patient.

In one embodiment, a PTH therapy, e.g., PTH or analogs thereof, is administered at a concentration from about 0.001-30, 0.05-30, 0.1-30, 1-30, 1-25, 1-20, 1-15, or 2-10 mg/kg body weight of the patient. In another embodiment, PTH, or an analog thereof, is administered at a concentration from about 2-10 mg/kg body weight of the patient. In one embodiment, PTH, or an analog thereof, is administered at a concentration between about 7 mg/kg body weight of the patient. In suitable embodiments, combinations of one or more proteasome inhibitors, such as bortezomib and/or carfilzomib, PTH, PTH analogs, and/or other PTHR1 activators, and/or other chemotherapeutic agents, are administered to a patient, separately, sequentially, or simultaneously, for the treatment of MM and variations thereof.

The therapeutic agents described herein may be administered in a variety of dosage forms. In some aspects, the present disclosure provides for compositions which may be prepared by mixing the therapeutic agents with pharmaceutically acceptable carriers, excipients, binders, diluents or the like to prevent skin cancer. Such compositions can be in the form of, for example, granules, powders, tablets, capsules, syrup, suppositories, injections, emulsions, elixirs, suspensions or solutions. The instant compositions can be formulated for various routes of administration, for example, by oral administration, by topical administration, by nasal administration, by rectal administration, subcutaneous injection, intravenous injection, intramuscular injections, or intraperitoneal injection. The following dosage forms are given by way of example and should not be construed as limiting the instant invention.

Treatment may also include administering the pharmaceutical formulations of the present disclosure in combination with other therapies. For example, the therapeutic agents, compounds, treatments, therapies, drugs, and/or pharmaceutical formulations, of the present disclosure may be administered before, during, or after a surgical procedure and/or radiation therapy. The compounds described herein can also be administered in conjunction with other anti-cancer drugs. By anticancer drugs is meant those agents which are used for the treatment of malignancies and cancerous growths by those of skill in the art such as oncologists or other physicians. Thus, anti-cancer drugs and compounds disclosed herein may be administered simultaneously, separately or sequentially. Appropriate combinations and administration regimes can be determined by those of skill in the oncological and/or medicinal arts.

### EXAMPLES

The present disclosure is further illustrated by the following examples, which should not be construed as limiting in any way. The following is a description of the materials and methods used throughout the examples.

Cell lines.

5TGM1 myeloma cells (Noboru et al., A multiresponse parathyroid hormone assay: an inhibitor has agonist properties in vivo. American Physiological Society. E589-95 (1983)) were initially derived from the myeloma cell line 5T33, which were obtained from C57BL/KaLwRij mice. See Dallas et al., Ibandronate Reduces Osteolytic Lesions but not Tumor Burden in a Murine Model of Myeloma Bone Disease. Blood. 93: 1697-1706 (1999). 5TGM1 cells were grown in Hyclone classical liquid media RPMI 1640 (Thermo Scientific Hyclone, Logan, Utah) supplemented with heat-inactivated fetal bovine serum (Thermo Scientific Hyclone, Logan, Utah) and 1% penicillin-streptomycin solution (GIBCO, Grand Island, N.Y.) at 37° C. in 5% CO₂. Additionally, two bortezomib-resistant MM cell lines, RPMI 8226 and ARP1, were generated via a stepwise increase in bortezomib (velcade) concentration. The IC₅₀ of RPMI8226 and ARP1 resistant-cells was determined to be 250.12 nM and 25.76 nM, respectively. The IC₅₀ of the parental (non-resistant) cells were 4.75 nM and 7.47 nM, respectively.

### Example 1

### Proteasome Inhibitors, PTH, and PTH inhibitors Effect MM Cell Survival

Analogs of PTH, such as {[TYR³⁴]bPTH-(7-34)}, also listed as anti-PTH or inhibitor of PTH (IPTH) herein, have been shown to be effective antagonists of PTH. See Noboru et al., A multiresponse parathyroid hormone assay: an inhibitor has agonist properties in vivo. American Physiological Society. E589-95 (1983). In this regard, {[TYR³⁴]bPTH-(7-34)} can potently antagonize PTH via stimulation of the renal-1 αhydroxylase, among other mechanisms, all of which are effected at comparable doses. Regardless of any such modes of action, the following results demonstrate that the PTHR1 pathway is a suitable target and/or biomarker for MM diagnosis, prognosis, and treatment.

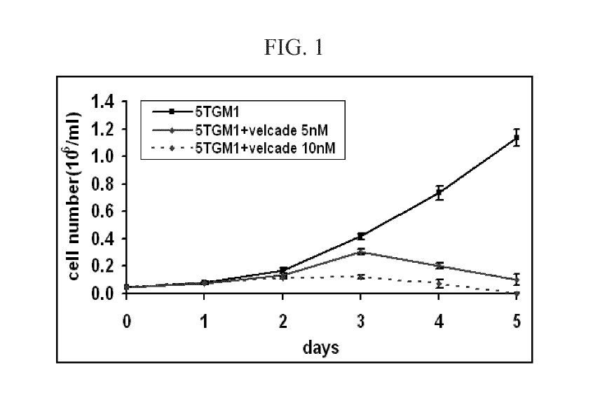

Initially, 5TGM1 myeloma cells were seeded in 24-well plates (105 cells/mL) using RPMI-1640, supplemented with 2% FBS, in the presence or absence of bortezomib (velcade), PTH, and/or {[TYR³⁴]bPTH-(7-34)}. Subsequently, 5TGM1 cells were challenged with bortezomib, PTH, and/or {[TYR³⁴]bPTH-(7-34)} at increasing concentrations and in various combinations, as listed below. As shown in FIG. 1, 5TGM1 control cells (growth medium only) were compared to 5TGM1 cells treated with bortezomib alone at 5 nM or 10 nM, every other day. The inhibitory effect of bortezomib is demonstrated by the concentration dependent reduction in 5TGM1 cell proliferation, compared to controls (p=0.01 at 5 nM; p=0.001 at 10 nM of bortezomib).

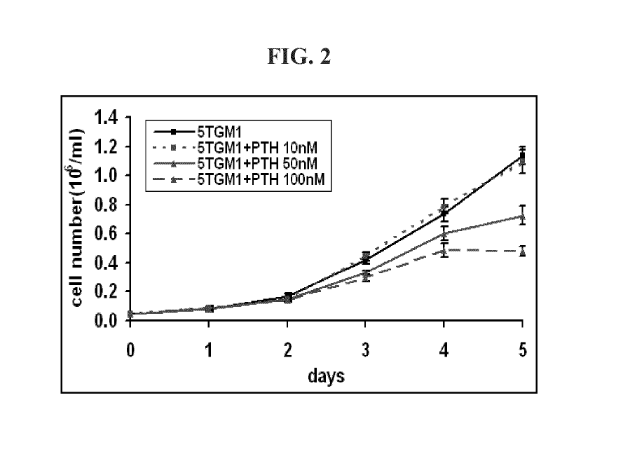

As shown in FIG. 2, 5TGM1 control cells (growth medium only) were compared to 5TGM1 cells treated with PTH alone at 10 nM, 50 nM, or 100 nM concentrations, once a day. A statistically significant decrease in cell growth was observed at the 50 nM and 100 nM PTH concentrations (p=0.001 and p=0.001, respectively). However, there was not a significant difference between control cells and cells treated with 10 nM of PTH (p=0.1). These results confirm that PTH exhibits a dose dependent inhibitory effect on 5TGM1 cell survival.

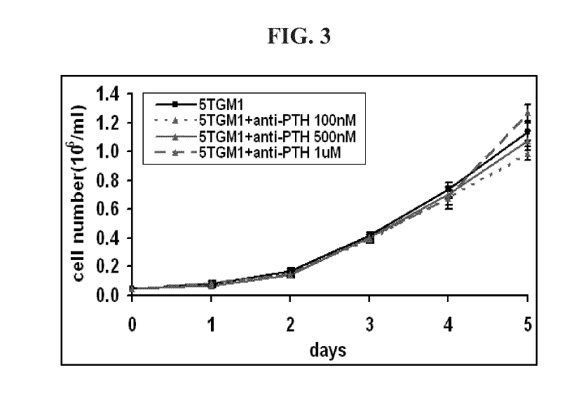

In view of the foregoing results, 5TGM1 cells were subsequently tested for their capacity to proliferate in the presence of absence of {[TYR³⁴]bPTH-(7-34)}, e.g., “anti-PTH”, exposure at increasing concentrations (FIG. 3). As shown in FIG. 3, 100 nM, 500 nM, and 1 μM concentrations of anti-PTH did not have an effect of 5TGM1 cell survival alone (p=0.1).

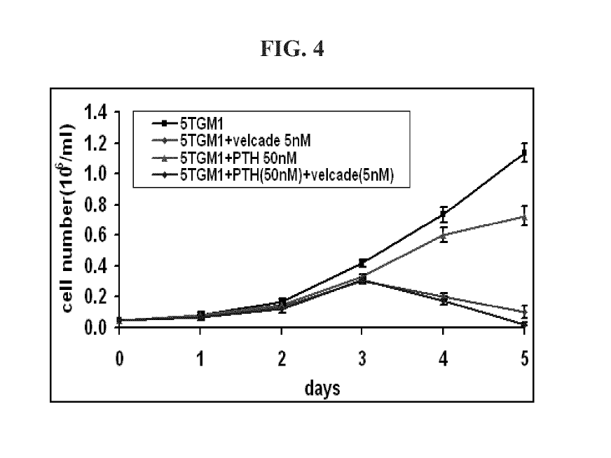

Next, the proliferative capacity of 5TGM1 cells were examined in the presence of bortezomib (velcade) and PTH (FIG. 4). The cells were treated with either bortezomib (5 nM) alone, PTH (50 nM) alone, or bortezomib (5 nM) and PTH (50 nM) together. Similar to the previous results, this combination exhibited an inhibitory effect on 5TGM1 cell growth, which was statistically significant compared to controls (p=0.001). Thus, these results demonstrate that the concomitant use of bortezomib, with PTH, did not effect the antiproliferative capacity of the former.

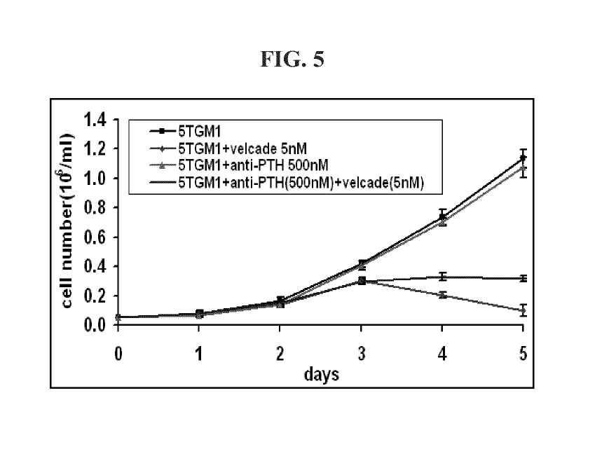

5TGM1 viability was also examined after exposure to bortezomib and {[TYR³⁴]bPTH-(7-34)} (anti-PTH). As shown in FIG. 5, 5TGM1 cells were treated with bortezomib (5 nM) alone, anti-PTH (500 nM) alone, or both bortezomib (5 nM) and anti-PTH (1 μM at 3 days and 1 day prior to bortezomib treatment). The inhibitory effect of bortezomib was decreased in the presence of the anti-PTH compound, which suggests that PTH receptor activation is important for induction of the bortezomib and/or PTH antimyeloma effect via proteasome inhibition (p=0.0001).

### Example 2

### Proteasome Inhibitors, PTH, and PTH inhibitors Posses In Vivo Efficacy

A preclinical myeloma model was employed to examine the effects of PTH and bortezomib (velcade) on proteasome activity with respect to bone remodeling and MM pathogenesis. As such, myelomatous C57BL/KaLwRij mice were injected with 5TGM1 multiple myeloma cells as described. See Zangari et al., Alkaline Phosphatase (ALP) Variation During Carfilzomib Treatment is Associated to Best Response in Multiple Myeloma. Blood. 114 Abstract 2865 (2009). Briefly, 5TGM1 inoculation cultures were harvested, centrifuged, washed twice in 50 mL phosphate-buffered saline (PBS), and resuspended at 5×10⁶ cells per mL of PBS. Experimental mice were injected with 100 μl of inoculation culture via tail vein injection using a 27-gauge needle. Control animals received injections of PBS.

Three weeks after 5TGM1 injection, myeloma disease was evident in the mice (data not shown). The mouse myeloma shared similar features to human myeloma, such as severe osteolysis and IgG production, in addition to terminal dispersion to non-bone organs including the liver and kidneys (data not shown). Since the 5TGM1/C57BL/KaLwRij mouse myeloma model closely mimics human MM, it was used to examine whether bortezomib, in the presence and absence of PTH, was efficacious in vivo. In this regard, the anti-PTH compound, {[TYR³⁴]bPTH-(7-34)}, was also tested in the presence or absence of bortezomib and PTH to determine whether myeloma growth and/or bone formation were affected. Similar experiments were performed using carfilzomib as shown below.

The overall survival of 5TGM1/C57BL/KaLwRij mice treated with bortezomib, PTH, and/or {[TYR³⁴]bPTH-(7-34)} was determined. The overall log rank test was employed for the data that follows, and indicated at least one statistically significant data point between treatment groups (p=0.0030). Pair wise log rank tests were also performed for each possible pair. Each experimental group included 5 mice, and one week after 5TGM1 cell injection, bortezomib, PTH, and/or {[TYR³⁴]bPTH-(7-34)} were administered in various combinations. The following groups, each containing five mice, were analyzed: untreated controls; bortezomib-treated mice; PTH treated mice; PTH and bortezomib treated mice; and 5{[TYR³⁴]bPTH-(7-34)} and bortezomib treated mice.

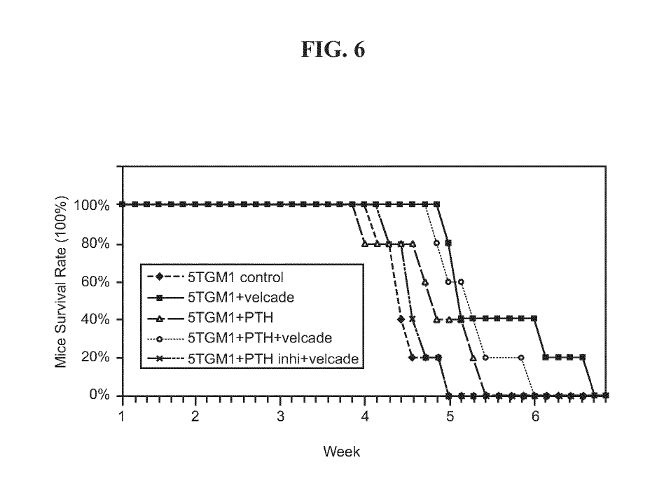

In accord with the experimental procedure outline above, bortezomib (velcade), at a concentration of 1 mg/kg, was administered to the mice via intraperitoneal injection alone, or in combination with PTH or the anti-PTH compound, twice a week (FIG. 6). PTH was added in a similar fashion, once-daily, alone, or in combination with bortezomib, at a concentration of 80 μg/kg. The anti-PTH inhibitor, {[TYR³⁴]bPTH-(7-34)}, was injected into the mice at a concentration of 500 μg/kg, alone, and in combination with bortezomib (1 mg/kg), where noted (FIG. 6). Along these lines, mice were treated with the anti-PTH compound 3 days prior, 2 days prior, 1 day prior, and immediately prior to proteasome inhibitor treatment. As shown in FIG. 6, bortezomib treated mice have a prolonged survival compared to controls (p=0.044). Control mice survived for a maximum of five weeks, whereas bortezomib treated mice survived for up to seven weeks, resulting in a 40% increased survival. Mice treated with PTH and bortezomib similarly showed a statistically significant increase in survival (p=0.044).

However, when mice were subjected to bortezomib and anti-PTH treatment, the beneficial effects of the proteasome inhibitor were completely abrogated (FIG. 6). To this point, mice treated with bortezomib survived for a significantly longer period of time compared to mice treated with bortezomib and the anti-PTH compound (p=0.044). A statistically significant survival was also observed in bortezomib and PTH treated mice compared to mice administered bortezomib with the anti-PTH compound (p=0.047).

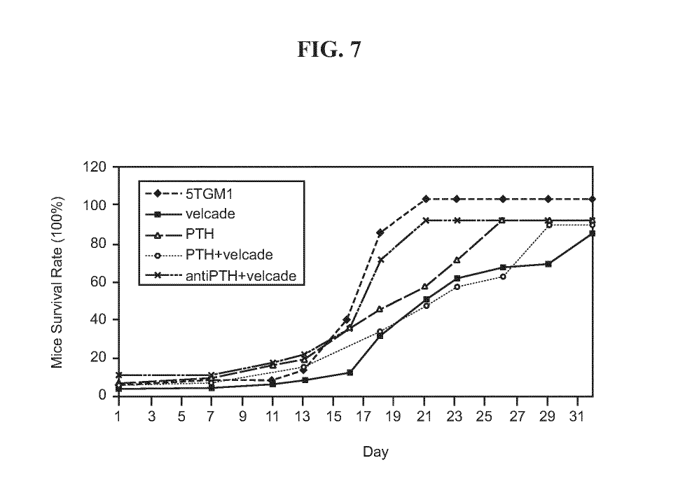

Congruent results were obtained when serum IgG2b levels were assessed for each mouse cohort (FIG. 7). To this end, bortezomib alone, PTH alone, and the combination of both, produced a significant inhibition of serum IgG level when compared to controls (p=0.017, p=0.029, and p=0.017, respectively). These results demonstrate that bortezomib and PTH are efficacious for inhibiting myeloma paraprotein secretion. Moreover, serum IgG levels for mice treated with bortezomib and the anti-PTH compound were significantly higher compared to IgG levels with bortezomib alone (p=0.034). These results demonstrate the abrogatory effects of the anti-PTH compound, {[TYR³⁴]bPTH-(7-34)}, on bortezomib induced paraprotein inhibition.

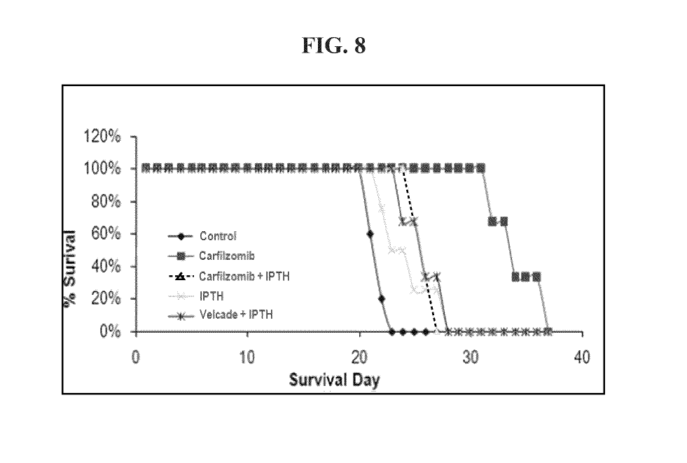

Similar to the experiments performed in the presence of bortezomib, the second generation proteasome inhibitor, carfilzomib, was also assessed in vivo. In these experiments, one week after 5TGM1 cell injection, mice were treated with carfilzomib, PTH, or the anti-PTH compound alone, or in the combinations shown (FIG. 8). C57BL/KaLwRij mice (n=5) were subcutaneously injected carfilzomib (1.5 mg/kg) twice a week. The anti-PTH compound (500 μg/kg) was also administered to the mice 3 days prior and 1 day prior to carfilzomib treatment, where noted. Tumor burden was monitored by measuring IgG2b serum concentration twice a week.

As shown in FIG. 8, carfilzomib treated mice have an increased survival compared to controls (p=0.012). Furthermore, mice treated with carfilzomib and the anti-PTH compound showed a statistically significant decrease in survival compared to mice treated with carfilzomib alone (p=0.024). These observations indicate that PTHR1 activation is linked to carfilzomib induced myeloma inhibition.

Accordingly, the foregoing in vivo results comport with the in vitro studies discussed above. In this regard, it was demonstrated that each proteasome inhibitor, bortezomib and carfilzomib, had positive effects on survival. These antimyeloma effects were also observed in combination with PTH. However, administration of the anti-PTH compound, {[TYR³⁴]bPTH-(7-34)}, with bortezomib or carfilzomib, nullified the effects of the protease inhibitors. These results were confirmed by serum IgG analysis. In short, the data presented herein indicates that myeloma proliferation is stymied in the presence of proteasome inhibitors, and that the inhibition is dependent upon the PTH-PTHR1 pathway. Consequently, PTHR1 expression and activation is capable of serving as a reliable biomarker and target for PTH, PTH analogs, and proteasome inhibitors, for the diagnosis, prognosis, and/or treatment of MM.

### Example 3

### PTHR1 Expression Correlates with Overall and Event-Free Survival in Patients Treated with Proteasome Inhibitors

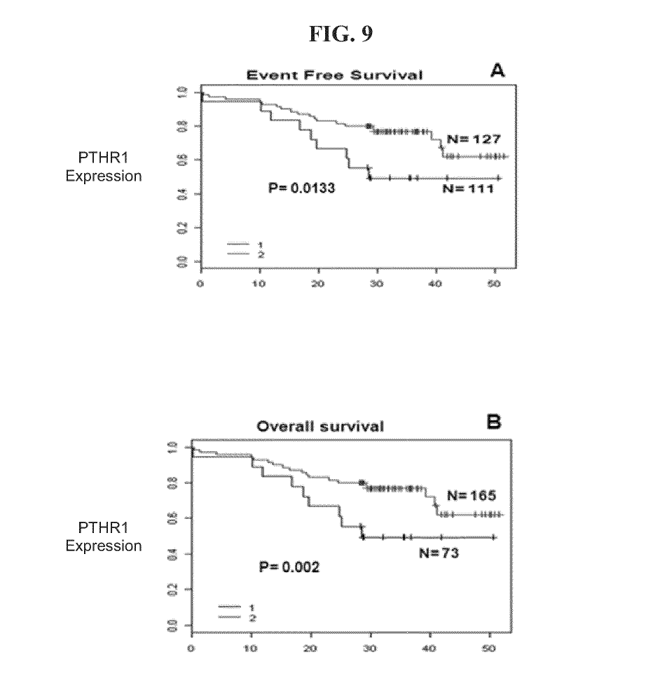

PTHR1 tissue expression was analyzed via gene array from unselected bone marrow core biopsies in order to further confirm that the PTH-PTHR1 pathway is essential for proteasome inhibitor-induced myeloma control. In this regard, the overall and event free survival of 238 mM patients were analyzed. As shown in FIG. 9, PTHR1 expression correlates with an increase in event free (FIG. 9A) and overall (FIG. 9B) survival for patients treated with proteasome inhibitors.

Treatment regimes included two cycles of VTD-PACE chemotherapy (bortezomib, thalidomide, dexamethasone and 4-d continuous infusions of cis-platin, doxorubicin, cyclophosphamide, etoposide) as induction before melphalan-based tandem transplantation. Consolidation chemotherapy consisted of two cycles of VTD-PACE at reduced doses. Thalidomide and dexamethasone were given to “bridge” drug-free intervals between induction cycles, and between transplantations and consolidation cycles. Maintenance therapy comprised monthly cycles of VTD (bortezomib, thalidomide and dexamethasone) in year one and TD (thalidomide and dexamethasone) in years two and three. See Barlogie et al., Incorporating bortezomib into upfront treatment for multiple myeloma: early results of total therapy 3. BJH. Vol. 138, p. 176-185 (2007).

Compared to basal PTHR1 expression, the results show a statistically significant increase in PTHR1 expression, which was associated with an increase in event free (p=0.001) and overall survival (p=0.002) for patients who received at least one year of bortezomib treatment (FIGS. 9A and 9B, respectively). This further demonstrates that PTHR1 expression levels are prognostic for increased patient survival when such patients are treated with proteasome inhibitors.

Unless otherwise defined, all technical and scientific terms used herein have the same meaning as commonly understood by one of ordinary skill in the art to which this invention belongs. All nucleotide sequences provided herein are presented in the 5′ to 3′ direction.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present disclosure has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

What is claimed is:

1. A method for determining a diagnosis, prognosis, or treatment regime for multiple myeloma in a subject, the method comprising: detecting a level of PTHR1 expression in a test sample from the subject, wherein a difference in the level of PTHR1 expression in the subject compared to a reference level is an indication of the subject's responsiveness to therapy selected from the group consisting of one or more proteasome inhibitors, PTH, and PTH analogs, or any combination thereof.

2. The method of claim 1, wherein the difference is an increase in the level of PTHR1 expression in the subject compared to the reference level and the increase indicates that the multiple myeloma is susceptible to the therapy selected from the group consisting of one or more proteasome inhibitors, PTH, and PTH analogs, or any combination thereof.

3. The method of claim 2, wherein the one or more proteasome inhibitors is selected from the group consisting of Bortezomib, Disulfuram, Salinosporamide A, Carfilzomib, CEP-18770, and MLN9708, or any combination thereof.

4. The method of claim 1, wherein the difference is an increase in the level of PTHR1 expression in the subject compared to the reference level and the increase is prognostic for an improved overall survival of the subject undergoing the therapy, compared to individuals afflicted with multiple myeloma that do not have the increase in the level of PTHR1.

5. The method of claim 1, wherein the difference is a decrease in the level of PTHR1 expression in the subject compared to the reference level and the decrease indicates that the multiple myeloma is resistant to the therapy selected from the group consisting of one or more proteasome inhibitors, PTH, and PTH analogs, or any combination thereof.

6. The method of claim 1, wherein the difference is a decrease in the level of PTHR1 expression in the subject compared to the reference level and the decrease is prognostic for a diminished overall survival of the subject compared to individuals that do not have the decrease in the level of PTHR1.

7. The method of claim 1, wherein the difference is a decrease in the level of PTHR1 expression in the subject compared to the reference level and the decrease is an indication that the multiple myeloma is refractory multiple myeloma.

8. The method of claim 1, wherein the subject has previously been treated for multiple myeloma or has previously been diagnosed with multiple myeloma.

9. The method of claim 1, wherein the reference level is the level of PTHR1 expression in a comparable sample from one or more healthy individuals.

10. The method of claim 1, wherein the detecting comprises amplifying a fragment of the PTHR1 mRNA.

11. The method of claim 10, wherein the amplifying is by polymerase chain reaction (PCR) or RT-PCR.

12. The method of claim 10, wherein the amplifying employs a detectably-labeled primer or probe.

13. The method of claim 1, wherein the detecting comprises measuring the presence, absence, or amount of a PTHR1 protein in the test sample from the subject.

14. The method of claim 13, wherein the measuring uses an antibody that specifically binds to the PTHR1 protein.

15. The method of claim 14, wherein the measuring is by an ELISA assay, a western blot assay, or an immunohistochemical assay.

16. The method of claim 1, wherein the test sample is a blood, serum, or biopsy sample.

17. The method of claim 1, wherein the subject is a human patient having or suspected of having multiple myeloma, refractory multiple myeloma, or has relapsed to having multiple myeloma.
